(19) United States
(12) Patent Application Publication
(10) Pub. No.: US 2008/0039949 A1
(43) Pub. Date: Feb. 14, 2008

(54) Artificial Joint Element And Gripping Tool Equipped With The Same

(76) Inventors: Larissa Meesenburg, Gottingen (DE); Hans Nagerl, Gleichen (DE)

Correspondence Address: ARNOLD INTERNATIONAL, P. O. BOX 129, GREAT FALLS, VA, 22066-0129, US

(21) Appl. No.: 11/792,049

(22) PCT Filed: Nov. 30, 2005
(86) PCT No.: PCT/DE05/02162

§ 371 Date: Oct. 15, 2007

(30) Foreign Application Priority Data
Dec. 3, 2004 (DE) 10 2004 058 546.6
May 2, 2005 (DE) 10 2005 020 779.0

(51) Int. Cl. A61F 2/42

(52) U.S. Cl. 623/21.11; 623/21.15

(57) ABSTRACT

The invention relates to an artificial joint element (1) for use in one or more single joints in a system of joints that consists of a combination of several kinematically coupled single joints, (series connection), in particular for use in one or more human finger joints. Said element comprises an essentially concave joint shell (2) and an essentially convex joint head (3). The aim of the invention is to obtain displacement characteristics for the artificial joint element (1), resulting from the additional degrees of freedom that are produced by incongruence, that mimic the natural behavior in an optimal manner. To achieve this, the joint element (1, 6) comprises at least four degrees of freedom and when used in the metacarpophalangeal joint, five degrees of freedom. For this purpose, the concave joint shell (2) comprises a curved contact line running over its surface with a first radius (R) and the concave joint head (3) has a contact line that is also curved running over its surface with a different second radius (r), which is smaller than the radius (R) of the contact line of the joint shell (2).

The invention relates to an artificial joint element for use in a human finger joint, with an essentially concave joint shell and with an essentially convex joint head. Furthermore, the invention relates to a gripping tool equipped with such a joint element.

Such an artificial joint element is known for example from U.S. Pat. No. 5,674,297 A which describes an artificial finger joint with a convex joint head and with a concave joint shell which independently of one another can be attached with a shaft in the end of a bone and are movable in an articulation plane from an extension position with parallel shaft axes into a hyperextension position or in an articulation end position. The joint head and the joint shell form a hinged joint here.

Furthermore, U.S. Pat. No. 4,231,121 shows a finger joint which consists of a joint head made as a ball head with attached shaft and a ball socket-shaped joint shell with a shaft likewise molded theron. The joint head and joint shell thus have a common articulation surface.

An artificial joint element is also the subject matter of EP 11 360 47 A1 which describes an artificial finger joint with a convex joint head and a concave joint shell. The joint head and the joint shell can be attached independently of one another in one end of the bone by means of a shaft and can be moved out of an extension position with parallel shaft axes into a hyperextension position or into an articulation end position. By attaching a second stop in the position in which a first stop for the hyperextension position is reached and in which an instantaneous center for continuation of rotation would form, movement is stopped and thus sliding of the joint head off the joint shell is avoided.

DE 43 37 922 A1 discloses a finger joint prosthesis, from whose joint shell a pin projects which on its free end bears a ball which forms an articulation surface with the joint shell. The ball is encompassed by the shell for purposes of locking in order to prevent unwanted laterally sliding.

EP 07 36 293 A 1 furthermore describes an artificial finger joint with a first and a second hollow anchoring projection which can be inserted into the tubular finger bone. Between the anchoring projections there is a hinge joint in the form of a ball joint, the first anchoring projection being connected to the ball cage of the hinged joint, in which a joint ball provided with another anchoring projection is supported. The anchoring projection passes through the slot of the ball cage and is connected to the second anchoring projection, the slot in the ball cage widening continuously in the extension position of the joint to the location at which the anchoring projection passes through the ball cage in the bending position of the joint.

DE 90 05 372 U1 also discloses a joint part prosthesis which is intended especially for a finger joint, with a bearing body which has a bearing surface and a shaft which can be embedded in the bone. The bearing body is made as a thin-walled shell element of high-strength metallic material with a thickness of a maximum 1 mm. The bearing surface functionally mimics that of a human finger joint in idealized form, and similar to a natural finger joint allows small lateral movements relative to the other joint part, in addition to the bending motion.

Furthermore, DE 196 50 816 A1 relates to an artificial finger joint. In order to move the finger again into the extended position with a tear of an extension ligament, the joint parts which are connected to the bone are connected by means of a flexible connecting element which develops a restoration force which opposes the bending.

In all known existing artificial joint elements the disadvantage is the associated movement capacity of the joint which can simulate natural joint properties only to a limited degree. Due to the differing joint properties, the artificial joint, due to the associated movement process, is often perceived by the patient as a foreign object and therefore as bothersome.

DE 102 31 538 C1 discloses an artificial joint, comprising a first joint compartment which is formed by a first joint head and a first joint socket, a second joint compartment which is formed by a second joint head and a second joint socket, the contact surfaces of the respective joint compartments in the primary function plane having an offset. In order to improve the properties of the artificial joint, the contact surfaces of the two joint compartments are configured tilted such that the surface normals of the contact surfaces have a common intersection point for each angle of flexion.

Therefore the object of the invention is to devise a possibility for creating a joint element which more or less approaches natural motion, comprising a joint shell and a joint head, in particular the degrees of the freedom which essentially determine the dynamic behavior are to be matched as required. Furthermore the object of the invention is to devise a gripping tool which is equipped with this artificial joint element.

The first object is achieved according to the invention with an artificial joint element according to the features of claim 1. Dependent claims 2 to 10 relate to especially advantageous developments of the invention.

According to the invention, there is therefore an artificial joint element in which the joint element has at least four degrees of freedom for use in a proximal interphalangeal joint (PIP) or in a distal interphalangeal joint (DIP), and especially for use in a metacarpophalangeal joint (MCP), has five degrees of freedoms. The invention is based on the finding that the properties of movement of the artificial joint can be caused to optimally approach natural behavior when the artificial joint allows additional degrees of freedom and thus clearly divergent kinematic coupling by way of the degrees of freedom which can be accomplished with the hinged joint known in the prior art.

In this connection, according to one practical embodiment the concave joint shell has a first curved contact line which runs on its surface, with a first radius, and the convex joint head has a second curved contact line which runs on its surface, with a second radius, the first radius of the first curved contact line at least in a partial region between the extreme locations of the joint position being larger than the second radius of the second curved contact line.

In this way there is therefore controlled incongruence of the joint head relative to the joint shell by which a contact region forms which is shaped especially as a line or point. Due to the additional degrees of freedom, moreover for especially impact-like loading peaks, damage to the artificial joint is avoided because a deflection movement of the joint is enabled. Moreover, due to the two angles of rotation which are implemented in this way significantly different force ratios arise due to the altered lever arm, so that even slight changes of the kinematics of movement lead to major changes of muscle forces. In particular, both a rolling motion around the contact point between the two contact lines as well as a sliding motion by the rotary motion around the center of the radius assigned to the joint shell or the joint head are thus conceivable. Furthermore, in conjunction with the synovial fluid which fills the gap, due to overflow or displacement of the fluid due to the change of the joint gap depending on the respective joint motion, an additional force component is produced which leads for example to a reduction of surface pressure and thus to improved wear behavior.

One especially advantageous embodiment of this invention is obtained in that the difference between the first radius and the second radius is at least 1 mm, especially between 2 mm and 4 mm. This results in optimum dynamic behavior due to the degrees of freedom which are determined by the difference of radii according to their amount, the difference of the radii of the MCP compared to the DIP and the difference of the radii of the PIP relative to the DIP increasing. Here the differences are especially dependent on the assignment to different finger joints, for the metacarpophalangeal joint (MCP) the difference of the radii is 3.8 mm, for the proximal interphalangeal joint (PIP) the difference of the radii is 1.3 mm, and for the distal interphalangeal joint (DIP) the difference of the radii is 1 mm.

An embodiment of this invention is also especially practical for which the difference between the first radius and the second radius in the bent joint position is larger than in the extended joint position so that the relative movement of the joint parts is determined by the respective angular position. In particular mobility is increased as a result of the increasing incongruence in the bent position. For this purpose, the convex joint head is composed of at least two ball segments which are made to meet one another in the region of a common tangent and thus enable continuous movement.

While the joint element to replace the MCP is made without concave regions, that is, in particular consists of a single joint head, conversely the joint head which is designed for use in PIP or DIP has two convex regions which are connected by a concavity. Deflection of the joint parts due to the external action of force from the relative desired position of the hinge head which is predefined for the respective joint position to the joint shell in conjunction with the tension force of the ligaments which initiate the motion leads to restoring motion into the desired position.

In this connection, according to one especially promising configuration of the artificial joint element the joint head and the joint shell each have two contact lines which are situated on the convex region between the concave connection region and the maximum distal extension, so that in particular the relative displacement of the joint parts in the transverse plane leads to a restoring force which counteracts the lifting of the contact region. The path of the contact line which deviates from the base which is enclosed by the two convex regions therefore leads to self-stabilization of the joint element designed in this way.

Furthermore, it is especially advantageous if the surface normals of the contact surfaces for each flexion angle have a common intersection point. This configuration of the contact surfaces yields self-stabilization of the joint both for initiation of rotational motion or torsion, and also for initiation of lateral forces.

The joint shell and the joint head could be made as a solid functional element with a connecting region for the finger bone. Conversely, a modification of this invention is especially advantageous in which the joint element is made as a thin molded part with a material thickness between 1 mm and 5 mm. In this way a major reduction in the loss of bone substance is achieved which moreover makes it possible to largely retain serviceable component regions of the natural joint.

Another likewise practical design is obtained, in contrast to use in human or animal joints, when a gripping tool equipped with at least one artificial joint according to one or more of the preceding claims can be actuated with a tension means which is connected by a drive unit. In this way a technical gripping tool is implemented which moreover allows simple control of movement and also a high reaction speed for transfer of a large force.

The invention allows different embodiments. To further illustrate its basic principle, one embodiment is shown in the drawings and is described below.

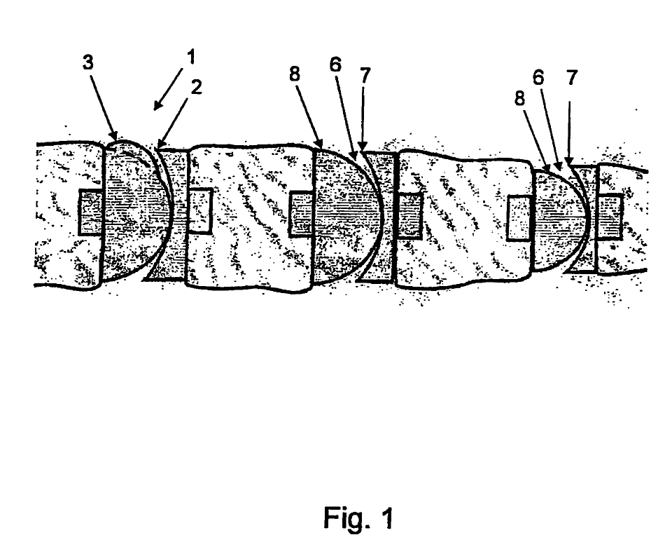

FIG. 1 shows a joint system with several kinematically coupled individual joints in a sagittal section;

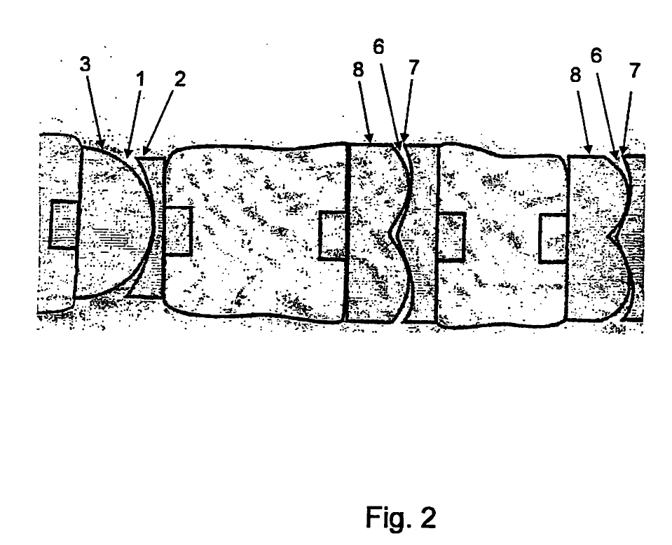

FIG. 2 shows the joint system shown in FIG. 1 in a horizontal section;

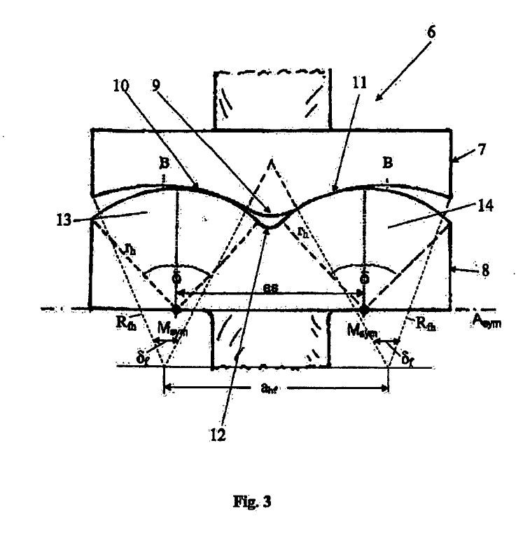

FIG. 3 shows a sagittal section of a joint element of the joint system shown in FIG. 1 in an enlargement;

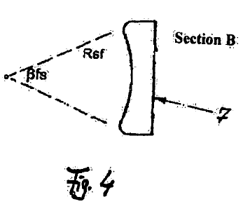

FIG. 4 shows a horizontal section of a concave joint shell of the joint element shown in FIG. 3;

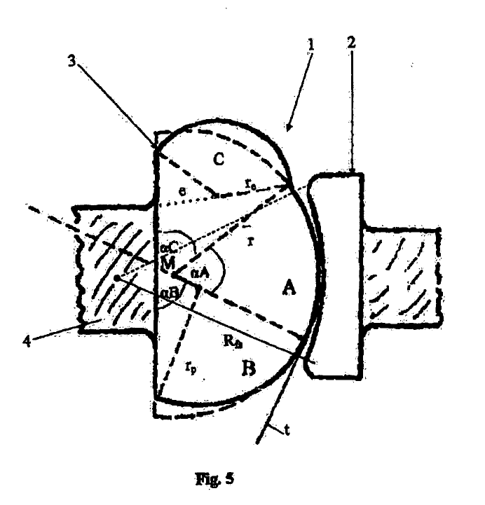

FIG. 5 shows a horizontal section of a joint element of the joint system shown in FIG. 1 in an enlargement;

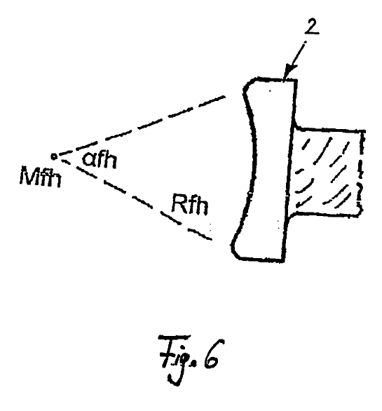

FIG. 6 shows a view of a concave joint shell of the joint element cut along line B in FIG. 5.

A joint system according to the invention for use of a human finger joint is detailed using the drawings, FIG. 1 showing a sagittal section and FIG. 2 shows a horizontal section of the joint system. This joint system comprises three kinematically coupled individual joints. Each of the joint elements 1 and 6 detailed in FIGS. 1 to 4 is equipped with an essentially concave joint shell 2, 7 and with an essentially convex joint head 3, 8 which each have at least four degrees of freedom due to their incongruence.

FIG. 3 shows in an enlargement the artificial joint element 1 of the joint system shown in FIG. 1 which is designed for replacement of a MCP, with its concave joint shell 2 and its convex joint head 3. The concave joint shell 2 has a curved contact line which runs on its surface with a first radius R_{fs}=10 mm and the convex joint head 3 has a likewise curved contact line which runs on its surface with a differing second radius r=7 mm±1 mm which is thus much smaller than the radius R_{fs} of the contact line of the joint shell 2. The joint head 3 underlies a spherical section which is formed by the joint head part A with a radius r and a projection 4 which is shown with a broken line. The joint head part A has a cut surface e which is connected to another spherical section as the joint head part C with radius rₑ=4 mm±1 mm. Opposite the cut surface e, a joint head part B is molded on with a tangent t of the respective surfaces of the joint head parts A and B, which tangent is identical in the transition region between the joint head part A and the joint head part B. Here the center line to a circle with a radius rₚ=6 mm±1 mm is formed. The amount of the angle for the joint head part A is the angle α_{A}=63°±5°, for the joint head part B the angle is α_{B}=65°±6° and for the joint head part C the angle is α_{c}=52°±5°. A simplified version shown by the broken line with identical radii r and rₚ of the joint head 3 can likewise be represented. The concave joint shell 2 is made as a more or less spherical shell with an oval function surface. For the radius R_{fs} of curvature thus the amount is more or less 10 mm and for the angle α_{fs} of the function surface the amount is 50°. These amounts should be understood as reference values and can vary by approx. 20%.

In addition, FIG. 4 shows a horizontal section of the concave joint shell shown in FIG. 3, with radius R_{fh}=9 mm and angle α_{fh}=53°, one modification with the amounts for the radius R_{fh}=9 mm and angle α_{fh}=50° likewise being feasible.

FIG. 5 shows in an enlargement a horizontal section of the artificial joint element 6 of the joint system shown in FIG. 2 designed for replacement of a PIP or DIP, with its concave joint shell 7 and its convex joint head 8. The joint shell 7 is provided with two concave function surfaces 10, 11 which are separated by a convexity 9 and which are each based on a body with rotational symmetry, for example on a barrel body or a sphere. The dimensions for the radius R_{hf} of the function surfaces 10, 11 of the joint shell 7 are 6.1 mm and have a distance a_{hf} of 7.3 mm. The angle δ_{f} of the respective function surfaces 10, 11 is identically 50° here. The two condyles 13, 14 of the joint head 6 which are separated by the concavity 12 are likewise based on a body with rotational symmetry to the axis of symmetry A_{sym}. The dimension of the radii rₕ=rₛ is 4.1 mm, the centers of the radii having a distance aₛ of 7.3 mm. Here a factor of approx. 0.7 should be prefixed to the indicated amounts of the radii, angles and distances for determining the amounts which are relevant for the DIP.

In addition, FIG. 6 shows a view of the concave joint shell 7 cut along line B-B in FIG. 5 with a radius R_{sf}=5.4 mm and an angle β_{pfs} of the function surface of 65°.

1. Artificial joint element (1, 6) for use of one or more individual joints in a joint system consisting of a combination of several kinematically coupled individual joints, especially for use in a human finger joint, each joint element (1, 6) being equipped with an essentially concave joint shell (2, 7) and with an essentially convex joint head (3, 8), characterized in that the joint element (1, 6) has at least four degrees of freedom.

2. The artificial joint element (1, 6) according to claim 1, wherein the joint element (1) for use in a metacarpophalangeal joint has five degrees of freedom.

3. The artificial joint element (1, 6) according to claim 1, wherein the concave joint shell (2, 7) has a first curved contact line which runs on its surface, with a first radius (R), and wherein the convex joint head (3, 8) has a second curved contact line which runs on its surface, with a second radius (r), the first radius (R) of the first curved contact line at least in a partial region between the extreme locations of the joint position being larger than the second radius (r) of the second curved contact line.

4. The artificial joint element (1, 6) according to claim 3, wherein the difference between the first radius (R) and the second radius (r) is at least 1 mm, especially between 2 mm and 4 mm.

5. The artificial joint element (1) according to claim 1, wherein the difference between the first radius (R) and the second radius (r) in the bent joint position is larger than in the extended joint position.

6. The artificial joint element (1) according to claim 1, wherein the artificial joint element (1) for replacement of the metacarpophalangeal joint (MCP) is made without concave regions.

7. The artificial joint element (6) according to claim 1, wherein the joint element (6) for replacement of the proximal interphalangeal joint (PIP) and/or distal interphalangeal joint (DIP) has a joint head (8) with two convex regions (condyles 13, 14) which are connected by a concave region (concavity 12).

8. The artificial joint element (1, 6) according to claim 7, wherein the joint head (6) and the joint shell (7) each have two contact surfaces which are situated in the convex region (condyles 13, 14) between the concave region (concavity 12) and the maximum distal extension.

9. The artificial joint element (1, 6) according to claim 1, wherein the surface normals of the contact surfaces have a common intersection point for each angle of flexion.

10. The artificial joint element (1, 6) according to claim 1, wherein the joint element (1, 6) is made as a thin molded part with a material thickness between 1 mm and 5 mm.

11. Gripping tool which is equipped with one or more artificial joint elements according to claim 1 and which can be actuated with a tension means which is connected by a drive unit, with an essentially concave joint shell and with an essentially convex joint head, the concave joint shell having a curved contact line with a first radius which is larger than the second radius of the second curved contact line of the convex joint head at least in one partial region.

12. The artificial joint element (1, 6) according to claim 2, wherein the concave joint shell (2, 7) has a first curved contact line which runs on its surface, with a first radius (R), and wherein the convex joint head (3, 8) has a second curved contact line which runs on its surface, with a second radius (r), the first radius (R) of the first curved contact line at least in a partial region between the extreme locations of the joint position being larger than the second radius (r) of the second curved contact line.

13. The artificial joint element (1, 6) according to claim 12, wherein the difference between the first radius (R) and the second radius (r) is at least 1 mm, especially between 2 mm and 4 mm.
